(19) United States
(12) Patent Application Publication
(10) Pub. No.: US 2014/0283184 A1
(43) Pub. Date: Sep. 18, 2014

(54) HYBRID CORN PLANT AND SEED S2338

(71) Applicant: Corn Products Development, Inc., Sao Paulo (BR)

(72) Inventors: Brad Ostrander, Zionsville, IN (US); Wade Ostrander, Indianapolis, IN (US)

(73) Assignee: Corn Products Development, Inc., Sao Paulo (BR)

(21) Appl. No.: 14/216,079

(22) Filed: Mar. 17, 2014

Related U.S. Application Data
(60) Provisional application No. 61/789,538, filed on Mar. 15, 2013.

(51) Int. Cl. A01H 5/10; C12N 15/82; A23L 1/10; A23L 1/0522

(52) U.S. Cl. CPC A01H 5/10; A23L 1/0522; C12N 15/8241; A23L 1/1041
USPC 800/263; 800/320.1; 800/303; 435/412; 800/275; 127/67; 127/29; 800/270; 426/443; 426/627; 426/622; 426/442

(57) ABSTRACT

This invention provides hybrid maize plant designated S2338. This invention further provides hybrid seed of S2338, hybrid plants produced from such seed, and variants, mutants, and trivial modifications to hybrid S2338, as well as methods of using the hybrid and products produced from the hybrid.

### CROSS-REFERENCE TO RELATED APPLICATIONS

This application claims the benefit of U.S. Provisional Patent Application No. 61/789,538, filed on Mar. 15, 2013, which is incorporated herein by reference in its entirety.

### FIELD OF THE INVENTION

This invention is in the field of maize breeding, specifically relating to waxy hybrid maize plants.

### BACKGROUND OF THE INVENTION

All publications and patent applications herein are incorporated by reference for all purposes to the same extent as if each individual publication or patent application was specifically and individually indicated to be incorporated by reference.

The following description includes information that may be useful in understanding the present invention. It is not an admission that any of the information provided herein is prior art or relevant to the presently claimed inventions, or that any publication specifically or implicitly referenced is prior art.

The goal of plant breeding is to combine in a single variety or hybrid various desirable traits, or to provide a desirable trait without significant detriment to other important properties. For field crops, desirable traits may include resistance to diseases and insects, tolerance to heat, cold and drought, reducing the time to crop maturity, greater yield, and better agronomic quality. With mechanical harvesting of many crops, uniformity of plant characteristics such as germination and stand establishment, growth rate, maturity, and plant and ear height is important. Other desirable traits may be those directly or indirectly associated with special nutritional and industrial types of crops. Examples of such specialty varieties or hybrids include those with higher oil content, different oil profiles, greater protein content, better protein quality or higher amylose content. It is also desirable to produce plants which are particularly adapted to a given agricultural region. New hybrids are an important part of efforts to control raw material costs.

Maize (Zea mays L.) is often referred to as corn in the United States, and the terms are used interchangeably in the present application. Maize has separate male and female flowers on the same plant, located on the tassel and the ear, respectively. Thus, it can be bred by crossing to itself (self-pollination or selfing), to another plant of the same family, line or variety (sib-pollination or sib-crossing) or to another plant of a different family, line or variety (outcrossing or cross-pollination).

Repeated self-pollination of plants, combined with selection for the desired type over many generations, results in inbred lines which are homozygous at almost all loci and thus will produce a uniform population of homozygous offspring when subject to further self-pollination. A cross between two different homozygous lines produces a uniform population of heterozygous hybrid plants. A cross of two plants each heterozygous at a number of gene loci will produce a population of heterogeneous plants that differ genetically and will not be uniform.

Hybrid maize varieties can be produced by a process comprising (1) the selection of plants from various germplasm pools for initial breeding crosses; (2) the selfing of the selected plants from the breeding crosses for several generations to produce a series of inbred lines as described above; and (3) crossing a selected inbred line with a different inbred line to produce the hybrid progeny (F1). Preferably, an inbred line should comprise homozygous alleles at about 95% or more of its loci.

Pedigree breeding and recurrent selection are two examples of methods used to develop an inbred line.

Pedigree breeding starts with the crossing of two or more genotypes, each of which may have one or more desirable characteristics. Superior progeny are selfed and selected in successive generations, during the course of which the level of homozygosity is increased. An inbred line suitable for hybrid production may be produced after a number of generations of selfing and selection, for example after four, five, six or more generations.

Double haploid methods can reduce the number of generations needed to obtain an inbred line. These methods involve the doubling of haploids derived from either the maternal or paternal gametes. Genetics markers can be used to identify haploids, and the haploids doubled to form homozygous diploid lines.

Recurrent selection entails individual plants cross-pollinating with each other to form progeny which are then grown. The superior progeny are then selected by any number of methods, which include individual plant, half sib progeny, full sib progeny, selfed progeny and toperossing. The selected progeny are cross pollinated with each other to form progeny for another population. This population is planted and again superior plants are selected to cross pollinate with each other. The objective of this repeated process is to improve the traits of a population. The improved population can then be used as a source of breeding material to obtain inbred lines to be used in hybrids.

Backcrossing can be used to improve inbred lines and a hybrid which is made using those inbreds. Backcrossing can be used to transfer a specific desirable trait from one line, the donor parent, to an inbred called the recurrent parent which has overall good agronomic characteristics yet that lacks the desirable trait. This transfer can be achieved by first crossing the recurrent parent with the donor parent, and then performing a backcross in which the progeny are mated to the recurrent parent. The resultant progeny can then be selected for the desired trait, and a further backcross performed using the selected individuals. Typically after four or more backcross generations with selection for the desired trait in each generation, the progeny will contain essentially all genes of the recurrent parent except for the genes controlling the desired trait. The last backcross generation is then selfed to give pure breeding progeny for the gene(s) being transferred.

Other plant breeding techniques known in the art, such as restriction fragment length polymorphism enhanced selection, genetic marker enhanced selection and transformation, may also be used in the production of inbred lines. For example, selection in the breeding process can be based upon the accumulation of markers linked to the positive effecting alleles and/or the elimination of markers linked to the negative effecting alleles from the plant's genome. Often, a combination of techniques is used.

For a review of plant breeding methods well known to those skilled in the art, see, for example, Sprague and Dudley (eds.), Corn and Corn Improvement, Third Edition, American Society of Agronomy, Inc., 986 pages, 1988; Fehr and Hadley (eds.), Hybridization of Crop Plants, American Society of Agronomy, Inc., 765 pages, 1980; Allard, Principles of Plant Breeding, John Wiley & Sons, Inc., 485 pages, 1960; Jensen, Plant Breeding Methodology, John Wiley & Sons, Inc., 676 pages, 1988; Simmonds, Principles of Plant Breeding, Longman Group Limited, 408 pages, 1979; and Hallauer and Miranda, Quantitative Genetics in Maize Breeding, Iowa State University Press, 468 pages, 1981.

In producing a hybrid strain by crossing two different inbred lines, it is advantageous to minimize the possibility of self-pollination. Minimizing self-pollination will minimize the proportion of the resultant seed which is substantially identical to the inbred line (resulting from the self-pollination) and increase the amount of hybrid seed (resulting from cross pollination). To this end, commercial maize hybrid production uses a male sterility system to render the female parent male sterile. There are several ways in which a maize plant can be manipulated so that it is male sterile. These include use of manual or mechanical emasculation (or detasseling), cytoplasmic genetic male sterility, nuclear genetic male sterility or gametocides (chemical agents affecting cells critical to male fertility, for example as described in Carlson, Glenn R., U.S. Pat. No. 4,936,904).

In detasseling, alternate strips of two inbred varieties of maize are planted in a field, and the pollen-bearing tassels are removed from one of the inbreds (female) prior to pollen shed. Providing that there is sufficient isolation from sources of foreign maize pollen, the ears of the detasseled inbred will be fertilized only from the other inbred (male), and the resulting seed is therefore hybrid and will form hybrid plants.

Alternatively, the female line can be cytoplasmic male sterile as a result of an inherited factor in the cytoplasmic genome. This characteristic is inherited exclusively through the female parent in maize plants, since only the female provides cytoplasm to the fertilized seed. CMS plants are fertilized with pollen from another inbred that is not male-sterile. Pollen from the second inbred may or may not contribute genes that make the hybrid plants male-fertile. The same hybrid seed, a portion produced from detasseled fertile maize and a portion produced using the CMS system can be blended to insure that adequate pollen loads are available for fertilization when the hybrid plants are grown.

Genetic male sterility may be conferred by one of several available methods, such as multiple mutant genes at separate locations within the genome that confer male sterility, as disclosed in U.S. Pat. Nos. 4,654,465 and 4,727,219 to Brar et al. and chromosomal translocations as described by Patterson in U.S. Pat. Nos. 3,861,709 and 3,710,511. A system in which male fertility genes are expressed under an inducible promoter is described in Albertsen et al., U.S. Pat. No. 5,432,068. Other approaches include delivering into the plant a gene encoding a cytotoxic substance associated with a male tissue specific promoter, or an antisense system in which a gene critical to fertility is identified and an antisense to that gene is inserted in the plant (see Fabinjanski, et al. EPO 89/3010153.8 publication no. 329,308 and PCT application PCT/CA90/00037 published as WO 90/08828).

Having obtained a desirable hybrid strain by the crossing of two different parent inbred strains, it is possible to maintain a uniform supply of the hybrid seed. The population of parent plants can be maintained by repeated self pollination. Moreover, since the parents are homozygous, the hybrid produced in the cross will always be the same. Thus, once a desirable hybrid has been identified, a continual supply of hybrid seed having the same properties can be provided.

Cytoplasmic male sterility can be used to facilitate the production of hybrid corn. For example, in one such scheme, hybrid corn is generated from four parental inbred corn lines: [parent A (S rf/rf)×parent B (N drf/rf)]=single-cross plant AXB (S rf/rf) which is crossed to [parent C (S rf/rf)×parent D (N Rf/Rf)]=single-cross plant CXD (S Rf/rf) to produce double-cross seed (A×B)×(C×D), wherein S rf/rf=male sterile and S Rf/rf=male fertile. The double-crossed hybrids are generally produced in an isolated field and subsequently planted to produce high-yielding double-cross corn plants. At each step, appropriate combinations of cytoplasmic genes and nuclear restorer genes ensure that the female parents will not self and that male parents will have fertile pollen. (J. Janick et al., Plant Science. 1974, W. H. Freeman and Company.)

Objectives of commercial maize hybrid line development include the development of new corn hybrids which are able to produce high yield of grain, which require less investment of time or resources, which are more resistant to environmental stresses (e.g., stresses particular to a certain growing area), which are easier to harvest and/or which provide grain or other products particularly suitable for a desired commercial purpose. To obtain a new hybrid, the corn breeder selects and develops superior inbred parental lines for producing hybrids. This is far from straightforward in view of the number of segregating genes and in view of the fact that the breeder often does not know the desired parental genotype in detail. Then, the breeder must identify the particular cross-combination of inbred lines which produces a desired hybrid. Even having obtained two superior inbred lines, there is no guarantee that the combination of these will produce desirable hybrid F1 plants. This is particularly the case because many selectable traits (e.g., yield) are dependent on the effects of numerous genes interacting with each other. Thus, the selection or combination of two parent lines produces a unique hybrid which differs from that obtained when either of the parents is crossed with a different inbred parent line.

### SUMMARY OF THE INVENTION

This invention relates to the development of a new waxy maize hybrid designated as S2338. S2338 is higher yielding than currently-grown waxy maize hybrids of similar maturity, type and adaptation. For example, S2338 yielded 7-9 bu/ac (bushels per acre) more than the mean yield of a current commercial hybrid (PPVO1864, U.S. Pat. No. 8,338,673). S2338 has comparable harvest moisture to other commercial waxy hybrids of similar maturity and is a soft grain type suitable for wet milling. S2338 further provides corn growers with a new waxy maize hybrid with high agronomic yield that is adapted to the central corn growing belt of the United States.

According to the invention, there is provided a novel corn hybrid, designated S2338, produced by crossing wsLH198 with wsLH324. Inbred lines wsLH198 and wsLH324 were proprietary lines developed by backcross breeding method, and are respectively the female and male parents of hybrid S2338. A representative sample of seed which when grown produces hybrid plants of S2338 is deposited under American Type Culture Collection (“ATCC”) accession number ______.

In some embodiments, instead of wsLH198, the female parent is wsLH198SDms, which is genetically identical or substantially identical to wsLH198 except that it possesses abnormal cytoplasm and is therefore male-sterile. SDms (a.k.a. Sdms) is a genetics-based breeding method for introducing cytoplasmic male sterility into corn plants which is well known by breeders, see e.g., Hallauer (Specialty Corns, Second Edition, 2001, CRC Press LLC, Table 5.6). This male-sterile line is maintained by backcrossing with wsLH198 which has the normal fertile cytoplasm. The hybrid produced by a cross between wsLH198SDms and wsLH324 is a sterile hybrid which is essentially identical to S2338, i.e., the sterile hybrid has the same plant genome of S2338, and the only difference is that it has the cytoplasmic mutation that leads to male-sterility. As used herein, the term “S2338” refers to both the fertile hybrid produced using wsLH198 and wsLH324 as the parents, and the sterile hybrid produced using wsLH198SDms and wsLH324 as the parents. S2338 provides corn growers with a new maize hybrid with high agronomic yield.

In one aspect, the present invention provides hybrid seed S2338, a representative sample of which has been deposited under ATCC accession number ______. The present invention also provides a population of corn seeds, wherein at least 80%, 81%, 82%, 83%, 84%, 85%, 86%, 87%, 88%, 89%, 90%, 91%, 92%, 93%, 94%, 95%, 96%, 97%, 98%, 99% or 100% of said seeds are hybrid seeds of which a representative sample has been deposited under ATCC accession number ______.

In another aspect, the present invention relates to a hybrid plant obtainable or obtained by growing seed of which a representative sample is deposited under ATCC accession number ______.

The present invention also provides hybrid plants having the plant genome of S2338. In some embodiments, the plants are male-sterile maize plants having the plant genome of S2338. In some embodiments, the male-sterile maize plant is produced by a cross of the two parents of S2338, wherein at least one parent is a SDms cytoplasmic male-sterile line.

The present invention also provides maize plants having all of the morphological and physiological characteristics of the present invention, such as S2338.

The present invention also provides tissue culture of regenerable cells produced from the plants of the present invention, or from the plant parts thereof. Also provided are maize plants regenerated from the tissue culture.

The present invention provides methods for producing maize seed. In some embodiments, the methods comprise crossing a maize plant of the present invention with itself or another maize plant, and harvesting the resultant seed. In some embodiments, S2338 is crossed with one or more maize inbreds, wherein the one or more maize inbreds are the same as one of the parents of S2338 (i.e., LH198 and/or LH 324) and/or are different than one of the parents of S2338. In some other embodiments, S2338 is crossed with itself and/or one or more maize hybrids that are different than S2338. In some embodiments, the different maize hybrids used in such crosses are one, two or more different waxy hybrids. Where the S2338 hybrid is crossed to another hybrid the invention produces double-cross hybrids. In some other embodiments, the S2338 male-sterile version is crossed to two other waxy hybrids used as male pollinators, wherein the resultant hybrid seed can be harvested from the S2338 female plants. In some embodiments, the methods comprise growing the resultant seed to produce one or more progeny maize plants. In some embodiments, the methods further comprising breeding from one or more of said progeny maize plants to produce progeny seed, and harvesting said progeny seed. In some embodiments, the methods further comprising growing said progeny seed, breeding from the resultant maize plants to produce seed, and harvesting said seed, over 1, 2, 3, 4, 5, 6 or more generations. Maize seeds and plants produced by any methods described herein are also within the scope of the present invention.

The present invention provides methods of producing a processed corn product. In some embodiments, the methods comprise providing at least one plant part of the present invention and processing said part(s) to produce a processed corn product. In some embodiments, the part is one or more corn kernels. In some embodiments, the processed corn product is corn starch, such as waxy corn starch. In some embodiments, the processed corn product is corn flour. A corn product produced by the methods described herein is also within the scope of the present invention.

The present invention provides methods for providing the processed corn product of the present invention. In some embodiments, the methods further comprise using said processed corn product in the production of a manufactured product. Modified waxy corn starches improve the uniformity, stability, and texture of food products. In some embodiments, the manufactured product is selected from the list consisting of a confectionery, a fried food product and a baked food product.

The present invention provides methods for producing a maize plant derived from hybrid S2338, or derived from a maize hybrid plant having the plant genome of S2338. In some embodiments, the methods comprise (a) crossing hybrid maize S2338 plant or the maize hybrid plant having the plant genome of S2338 with a second maize plant and harvesting the resultant maize seed, wherein representative seed of S2338 has been deposited under ATCC Accession Number ______. In some embodiments, the methods further comprise (b) growing said resultant maize seed to produce a maize plant derived from S2338, or derived from the maize plant having the plant genome of S2338. In some embodiments, the maize hybrid plant having the plant genome of S2338 is a male-sterile maize plant. In some embodiments, the male-sterile maize plant has the same parents of S2338, except for that the female parent is a male-sterile version of the female parent of S2338.

The present invention also provides methods for developing a maize plant in a plant breeding program using plant breeding techniques. In some embodiments, the methods comprise employing a maize plant, or its part, as a source of plant breeding material comprising using the maize plant, or its part, of the present invention as a source of breeding material.

The present invention also provides methods for developing a second maize plant in a plant breeding program comprising applying plant breeding techniques to a first maize plant, or parts thereof. In some embodiments, said first maize plant is the maize plant of the present invention and wherein application of said techniques results in development of said second maize plant. In some embodiments, said plant breeding techniques are selected from the group consisting of pedigree breeding, recurrent selection, backcrossing, restriction fragment length polymorphism enhanced selection, genetic marker enhanced selection and transformation. In some embodiments, the first maize plant of is S2338. In some embodiments, the first maize plant is a male-sterile maize hybrid plant having the plant genome of S2338. In some embodiments, the male-sterile maize plant has the same parents of S2338, except for that the female parent is a male-sterile version of the female parent of S2338. In some embodiments, the male-sterile version of S2338 is crossed with a fertile pollinator to produce the second maize plant. In some embodiments, the fertile pollinator is a waxy pollinator hybrid maize plant.

In some embodiments, the present invention provides waxy hybrid plants. Endosperm of waxy maize contained only amylopectin and no amylose starch molecule in opposition to normal dent maize varieties that contain both. Waxy starch is of main interest because fractionation of normal starch to obtain pure amylose or amylopectin is very costly. Waxy maize could produce more efficient feed gains than normal dent maize. Several unique structural features enable the plants to resist the drying out of the silks by wind at the time of flowering. Waxy maize plants also can have unusual growth behavior in that the top four or five leaves all appeared on the same side of the main stem of the plant. The plants may have extremely erected leaves of the upper nodes, while their lower leaves are more spread and drooping.

The starch of normal dent maize is characterized by a content of about 25% amylose with the remainder being amylopectin and the intermediate fraction. But these percentages vary among cultivars and with kernel development. For example, amylose percentage ranged from 20 to 36% for 399 cultivars of normal maize. There are maize germplasm collected that range from less than 20 to 100% complement of amylopectin (Fergason, 2001, High Amylose and Waxy Corns (pp. 63-84). Specialty Corns. A. R. Hallauer, Boca Raton, CRC Press: 479 pp).

For waxy maize, a single recessive gene (wx) was located on the short arm of chromosome 9, codes for the waxy endosperm of the kernel (wx codes for endosperm with normal starch). The waxy gene is epistatic for all known other amylose and amylopectine forming mutant genes. The wx locus is expressed in the endosperm, in the male gametophyte (pollen) as well as in the female gametophyte (embryo sac).

Amylopectin or waxy cornstarch is relatively easy to gelatinise, produces a clear viscous paste with a sticky or tacky surface. The paste rheology resembles pastes of root or tuber starches, such as potato starch or tapioca starch (made from cassaya). Amylopectine starch also has a lower tendency to retrogradate and is thus more viscosity stable. These different properties compared to normal dent corn starch, containing also amylose, are utilized mainly in following different applications.

The present invention also provides waxy maize starches produced from the hybrid plants or plants derived thereof. In some embodiments, the waxy maize starches are modified. Non-limiting modified waxy maize starches include, acid-treated starch, dextrin treated with hydrochloric acid, alkaline-modified starch, bleached starch, oxidized starch, breaking down viscosity enzyme-treated starch, monostarch treated with phosphorous acid or the salts sodium phosphate, potassium phosphate, or sodium triphosphate to reduce retrogradation, acetylated starch, hydroxypropylated starch, Octenyl succinic anhydride (OSA) starch, cationic starch, and carboxymethylated starch.

The invention also relates to variants, mutants and trivial modifications of the hybrid seed or plant.

Seeds, plants, plant parts, somatic tissues or cells according to the present invention may have substantially the same genotype as the deposited seed ATCC ______, and/or may be capable of serving as the source for tissue culture to produce a plant of substantially the same genotype as hybrid seed deposited under ATCC accession number ______.

In another aspect the present invention provides a corn plant (or seed thereof) having desirable traits of hybrid S2338. The corn plant may have all or essentially all of the morphological or physiological characteristics of hybrid S2338. Optionally, the plant may have one or more additional characteristics, e.g., characteristics resulting from the presence of one or more nucleic acid sequences introduced by techniques known to those skilled in the art, such as transgenic techniques or conventional breeding methods such as backcrossing. In some embodiments, such a corn plant is produced by crossing wsLH198SDms (male sterile, female parent) and wsLH324 (male parent). In some embodiments, the hybrid corn plants of the present invention include hybrid corn plants of S2338 which further include one, two, three or more foreign or heterologous genes introduced into S2338. Such foreign or heterologous genes may be from a different corn plant (i.e., a corn inbred, corn hybrid, corn haploid, etc.) other than the inbreds used to produce S2338, and/or from a plant species other than Zea mays (e.g., alfalfa, soybean, canola, tomato, potato, yew tree, marigold, etc.), and/or from a non-plant species (e.g., bacteria, fungi, insects, mammals, jellyfish, etc.).

The invention further relates to corn plants and seeds derived from hybrid maize plants of the present invention, such as the hybrid maize plant S2338. These plants and seeds may be of an essentially derived variety as defined in section 41(3) of the Plant Variety Protection Act, i.e., a variety that:

(i) is predominantly derived, or predominantly derived from a hybrid maize plant of the present invention, such as the hybrid maize plant S2338, while retaining the expression of the essential characteristics that result from the genotype or combination of genotypes of the hybrid maize plant of the present invention, such as hybrid S2338;

(ii) is clearly distinguishable from hybrid S2338; and

(iii) except for differences that result from the act of derivation, conforms to the initial variety in the expression of the essential characteristics that result from the genotype or combination of genotypes of the initial variety.

An essentially derived variety may be obtained by the selection of a natural or induced mutant or of a somaclonal variant, the selection of a variant individual from plants of hybrid S2338, backcrossing, transformation by genetic engineering, or any other method.

The essential characteristics may be one or more of the desirable traits set forth herein.

The corn plants and seeds derived from hybrid maize S2338 may in other embodiments be regenerated from a tissue culture produced from a hybrid S2338 plant, or may be a plant or seed having hybrid S2338 as an ancestor, as discussed further below.

The present invention also provides a tissue culture of regenerable cells produced from hybrid plant S2338, wherein said tissue culture is capable of producing plants having desirable traits of hybrid S2338 as set out above. The tissue culture may be derived directly or indirectly from hybrid S2338. Preferably the tissue culture is capable of producing plants which have all or substantially all of the morphological and physiological characteristics of hybrid S2338. Optionally, the plants may have one or more additional characteristic, e.g., conferred by a nucleic acid sequence introduced using transgenic or conventional breeding techniques. In some embodiments the plant may have the genetic complement of hybrid S2338, optionally comprising one or more additional nucleic acid sequences capable of modifying the phenotype of the plant when expressed (e.g., as RNA or protein). The culture can be from any tissue capable of somatic embryogenesis, e.g., may be selected from the group consisting of leaf, pollen, embryo, root, root tip, anther, silk, flower, kernel, ear, cob, husk, stalk, cell or protoplast.

The invention further relates to the use of the tissue culture to produce a whole plant, to protoplasts produced from said tissue culture and to a corn plant regenerated from said tissue culture. A method of producing a whole plant from the tissue culture may comprise one or more of: culturing cells in vitro in a media comprising an embryogenesis promoting hormone until callus organization is observed; transferring cells to a media which includes a tissue organization promoting hormone; after tissue organization is observed transferring cells into a media without said hormone to produce plantlets; and growing said plantlets, optionally including growing said plantlets on a minimal media for hardening.

In a further aspect of the present invention, there is provided pollen or an ovule of hybrid plant S2338, as well as seed produced by fertilization with said pollen or of said ovule, and plants grown from the seed.

The hybrid plant S2338 can be crossed with a corn plant of another line or variety, or can be sib-crossed or selfed to produce another plant, line (e.g., inbred line) or population of plants (e.g., breeding population of plants) which is of benefit in plant breeding.

Thus, in another aspect the present invention relates to a plant or seed produced by a breeding program using hybrid S2338 as a parent, wherein the plant or seed is a member of a generation of progeny of said parent, e.g., a member of the first, second, third, fourth, fifth, sixth or more generation of progeny. Thus, the present invention includes plants and seeds produced using hybrid S2338 as an ancestor. Ancestry can be assessed from the records kept routinely by one of ordinary skill in the art. It can also be assessed based on nucleic acid identity, e.g., using molecular markers, electrophoresis and the like. The plant or seed thus produced may have desired characteristics of hybrid S2338 as discussed above, or may have all of the morphological and physiological traits of hybrid S2338.

In another aspect the present invention relates to use of a hybrid S2338 maize plant to produce seed and/or progeny maize plants. The present invention also provides a method comprising providing a plant of hybrid S2338, crossing it with itself or with another maize plant (which may be another hybrid S2338 plant or may be a plant of a different line or variety) so as to produce seed, and harvesting said seed. The method may further comprise growing said seed to produce one or more progeny maize plants, and optionally, breeding from one or more of said progeny maize plants to produce progeny seed, which may be harvested. The step of growing the progeny seed and breeding from the resultant maize plants to produce a further population of seed can be repeated over one or more further generations (e.g., in 1, 2, 3, 4, 5, 6 or more further generations). For instance, the progeny may be selfed, sibbed, backcrossed, crossed to a population or the like. By “breeding from” a plant is meant a process of crossing the plant with itself or with another plant of the same or a different variety to produce seed. Selection may be carried out in one or more of the progeny generations. The selection may be for one or more desirable traits of hybrid S2338, e.g., one or more of amylose content of the starch and agronomic yield. Selection may be done using visual inspection, or using molecular markers.

Plants resulting from such methods would contain desirable traits derived from hybrid S2338 and thus would benefit from the work of the present inventors and from the disclosure contained herein.

For instance, in one embodiment, a method of the invention may comprise sib or self-pollinating hybrid S2338 to produce a first generation of progeny plants. The method may further comprise sib or self-crossing said progeny over one or more further generations (e.g., 1, 2, 3, 4, 5, 6 or more further generations) and/or double haploid breeding, in order to produce a plant which is substantially homozygous, e.g., greater than 90%, 91%, 92%, 93%, 94%, 95%, 96%, 97%, 98%, 99%, 99.1%, 99.2%, 99.3%, 99.4%, 99.5%, 99.6%, 99.7%, 99.8%, 99.9%, 99.95% or more homozygous. This method may comprise selection of plants having the one or more desirable traits of the parent plant. This selection may take place in each progeny generation or less frequently, e.g., in 1, 2, 3, 4, 5 or more generations of progeny (e.g., in the first progeny generation and/or in one or further progeny generations.

In another embodiment, a hybrid maize plant as described herein can also be crossed to a different variety of maize, such as an inbred line (e.g., an elite inbred line). The F1 progeny generation resulting from this cross would have 50% of its genes derived from the hybrid S2338. The method may further comprise self-fertilization of one or more plants from the F1 population to produce an F2 progeny generation. Some of the F2 plants will by chance have more than 50% of their genes derived from the parental hybrid plant. These may be selected, for example using molecular marker selection or selection of one or more desired traits of hybrid S2338. Self-fertilization of the progeny may be carried out over 1, 2, 3, 4, 5 or more further generations to produce an inbred line. Selection may be carried out in each progeny generation, or at a lower frequency, e.g., in 1, 2, 3, 4, 5 or more of the generations.

The method may in some embodiments further comprise modification of the resultant inbred line to provide a further desired trait or traits. For instance, the method may comprise crossing the resultant inbred line with a further plant variety having a desirable trait, and backcrossing the progeny over 1, 2, 3, 4, 5, 6 or more generations so as to insert the desired trait into a genetic background which is substantially that of the inbred line. In another embodiment, the method may comprise transgenic modification of the inbred line, which can be carried out using methods which would be well known to those in the art.

In a further embodiment the method comprises crossing a plant of a first variety or line to a plant of a second, different variety or line, wherein the first variety or line is hybrid S2338. The second variety or line may be an inbred line and in some embodiments, may be of one of the parental lines of hybrid S2338. The method may comprise growing a first progeny generation. The method may then further comprise backcrossing one or more plants of that progeny generation to one or more plants of the second variety or line to produce a further progeny generation. The backcrossing may be repeated in 1, 2, 3, 4, 5, 6 or more generations. The last backcross generation may be selfed to result in a pure breeding line for the desired trait(s). Selection may be carried out in one or more of the progeny populations, e.g., to select plants having one or more desirable traits of hybrid S2338.

The invention also includes the population of seeds or plants produced at any stage of the breeding methods described above. In some embodiments, the seed or plant may be an inbred seed or plant, e.g., such as may be used for a further breeding program or for the development of further hybrids.

Corn is a highly useful crop, and numerous commercial products can be provided by or derived from its different parts. Accordingly, the present invention provides use of a plant as described herein for the production of a processed corn product.

Also provided is a method comprising providing one or more parts of a plant as described herein and processing said part(s) to produce a processed corn product. The method may also comprise growing the plant and/or harvesting said one or more parts.

The plant part may be any of the parts described above, including the stem, husk or cob, shoot, root, seeds, stipules, leaves, ears, silk, tassel, stalk, pollen, ovules, petioles, internodes, pubescence, tillers, fronds, blades, sheath, whorl, and the like, but in many embodiments will be the ear or the kernels.

The invention also provides products derived from the corn plants described herein. Any and all products made using the seeds, plants and parts thereof obtained from the transgenic plants or from any line produced using the transgenic plants described herein as a direct or indirect parent are also part of the invention. Examples of processed corn products are corn starch (including isolated corn starch components such as amylose or amylopectin), flour, grits, meal, corn syrup or dextrose, corn oil, processed corn grain products such as canned, frozen or pureed grain, ethanol, paper, wall-board or charcoal. The origin of the corn used in such corn products can be determined by tracking the source of the corn used to make the products and/or by using protein (isozyme, ELISA, etc.) and/or DNA (RFLP, PCR, SSR, SNP, EST, etc.) testing.

For instance, in one embodiment the invention provides a method for the production of corn starch comprising providing kernels of a plant as described herein, and processing the kernels to produce corn starch. The processing may comprise wet-milling.

In another embodiment, the invention provides a method for the production of corn flour comprising providing kernels of a plant as described herein, and processing the kernels to produce corn flour. The processing may comprise dry-milling.

The invention also provides a method comprising, having provided a processed corn product as described above, using said processed corn product in the production of a manufactured product. These may be any of the manufactured products as described further below. Examples include a food product, packaging, adhesive, paper or textile, pharmaceutical product, cosmetic, and home care product.

The invention further provides a processed corn product or manufactured product produced by any of the methods described above. In some embodiments, the corn product is waxy corn starch or flour.

The invention also provides methods of producing hybrid maize seed comprising crossing a male-sterile version of S2338 with at least one waxy pollinator hybrid and harvesting the resultant seed from the male-sterile version of S2338. In some embodiments, the S2338 hybrid is crossed to one other maize hybrid to produce a double-cross hybrid. In some embodiments, the present invention provides such methods wherein the male-sterile version of S2338 is crossed with two waxy pollinator hybrids. In some other embodiments, the present invention provides such methods which include growing a hybrid maize plant from the hybrid maize seed harvested from the male-sterile version of S2338.

### DETAILED DESCRIPTION

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, the verb “comprise” as is used in this description and in the claims and its conjugations are used in its non-limiting sense to mean that items following the word are included, but items not specifically mentioned are not excluded.

As used herein, the term “a” or “an” refers to one or more of that entity; for example, “a gene” refers to one or more genes or at least one gene. As such, the terms “a” (or “an”), “one or more” and “at least one” are used interchangeably herein. In addition, reference to “an element” by the indefinite article “a” or “an” does not exclude the possibility that more than one of the elements are present, unless the context clearly requires that there is one and only one of the elements.

As used herein, the term “allele” refers to any of several alternative forms of a gene.

As used herein, “starch” refers to starch in its natural or native form as well as also referring to starch modified by physical, chemical, enzymatic and biological processes.

As used herein, “amylose” refers to a starch polymer that is an essentially linear assemblage of D-anhydroglucose units which are linked by alpha 1,6-D-glucosidic bonds.

As used herein, “amylose content” refers to the percentage of the amylose type polymer in relation to other starch polymers such as amylopectin.

As used herein, “area of adaptation” refers to an area having a particular combination of environmental conditions under which this corn hybrid will grow well. The term is not intended to mean that the corn hybrid will not grow outside of this region, particularly, that it will not grow equally well in areas sharing the same or substantially the same combination of conditions.

As used herein, “waxy maize” refers to the generic name for corn that expresses at least 99% to 100% amylopectin starch in the kernel.

As used herein, “waxy maize inbred” refers to maize inbred that expresses 100% amylopectin starch in the kernels.

As used herein, “waxy maize hybrid” or “waxy hybrid” refers to a hybrid whose grain produces 100% amylopectin starch.

As used herein, the terms “crossing” or “crossed” or grammatical equivalents thereof refer to pollen from one flower being transfers to the ovule of the same or a different flower to result in fertilization. A plant crossed to itself is self-pollinated or selfed; a plant crossed to another plant of the same variety, family or line is sib-pollinated or sib-crossed and a plant crossed to another plant of a different variety, family or line is out-crossed or out-pollinated.

As used herein, the term “cross pollination” or “cross-breeding” refer to the process by which the pollen of one flower on one plant is applied (artificially or naturally) to the ovule (stigma) of a flower on another plant.

As used herein, the term “cultivar” refers to a variety, strain or race of plant that has been produced by horticultural or agronomic techniques and is not normally found in wild populations.

As used herein, the term “elite inbred line” refers to an inbred which has been shown to contribute desirable qualities when used to produce commercial hybrids.

As used herein, the term “female” refers to a plant that produces ovules. Female plants generally produce seeds after fertilization. A plant designated as a “female plant” may contain both male and female sexual organs. Alternatively, the “female plant” may only contain female sexual organs either naturally (e.g., in dioecious species) or due to emasculation (e.g., by detasselling).

As used herein, the term “filial generation” refers to any of the generations of cells, tissues or organisms following a particular parental generation. The generation resulting from a mating of the parents is the first filial generation (designated as “F1” or “F₁”), while that resulting from crossing of F1 individuals is the second filial generation (designated as “F2” or “F₂”).

As used herein, the term “gamete” refers to a reproductive cell whose nucleus (and often cytoplasm) fuses with that of another gamete of similar origin but of opposite sex to form a zygote, which has the potential to develop into a new individual. Gametes are haploid and are differentiated into male and female.

As used herein, the term “gene” refers to any segment of DNA associated with a biological function. Thus, genes include, but are not limited to, coding sequences and/or the regulatory sequences required for their expression. Genes can also include nonexpressed DNA segments that, for example, form recognition sequences for other proteins. Genes can be obtained from a variety of sources, including cloning from a source of interest or synthesizing from known or predicted sequence information, and may include sequences designed to have desired parameters. Thus, this invention further encompasses the maize plants, and parts thereof, of the present invention which have been transformed so that its genetic material contains one or more transgenes operably linked to one or more regulatory elements. Furthermore, the maize plants, or parts thereof, of the present invention also encompass such maize plants, or parts thereof, that contain a single gene conversion.

As used herein, the term “genetic complement” refers to the complete set of alleles possessed by a cell. In a plant or other somatic tissue or cell the complement will be diploid—that is, there will be two alleles (the same or different) at each locus.

As used herein, the term “genotype” refers to the genetic makeup of an individual cell, cell culture, tissue, plant, or group of plants.

As used herein, the term “grain” refers to mature corn kernels produced by commercial growers for purposes other than growing or reproducing the species.

As used herein, the terms “heterologous polynucleotide” or a “heterologous nucleic acid” or an “exogenous DNA segment” refer to a polynucleotide, nucleic acid or DNA segment that originates from a source foreign to the particular host cell, or, if from the same source, is modified from its original form. Thus, a heterologous gene in a host cell includes a gene that is endogenous to the particular host cell, but has been modified. Thus, the terms refer to a DNA segment which is foreign or heterologous to the cell, or homologous to the cell but in a position within the host cell nucleic acid in which the element is not ordinarily found. Exogenous DNA segments are expressed to yield exogenous polypeptides.

As used herein, the term “heterologous trait” refers to a phenotype imparted to a transformed host cell or transgenic organism by an exogenous DNA segment, heterologous polynucleotide or heterologous nucleic acid.

As used herein, the term “heterozygote” refers to a diploid or polyploid individual cell or plant having different alleles (forms of a given gene) present at least at one locus.

As used herein, the term “heterozygous” refers to the presence of different alleles (forms of a given gene) at a particular gene locus.

As used herein, the term “homologue” refers to a nucleic acid or peptide sequence which has a common origin and functions similarly to a nucleic acid or peptide sequence from another species.

As used herein, the term “homozygote” refers to an individual cell or plant having the same alleles at one or more loci.

As used herein, the term “homozygous” refers to the presence of identical alleles at one or more loci in homologous chromosomal segments.

As used herein, the term “hybrid” refers to any individual cell, tissue or plant resulting from a cross between parents that differ in one or more genes.

As used herein, the term “inbred” or “inbred line” refers to a relatively true-breeding strain.

As used herein, the term “kernel” refers to the corn caryopsis comprising a mature embryo and endosperm which are products of double fertilization.

As used herein, the term “line” is used broadly to include, but is not limited to, a group of plants vegetatively propagated from a single parent plant, via tissue culture techniques or a group of inbred plants which are genetically very similar due to descent from a common parent(s). A plant is said to “belong” to a particular line if it (a) is a primary transformant (T0) plant regenerated from material of that line; (b) has a pedigree comprised of a T0 plant of that line; or (c) is genetically very similar due to common ancestry (e.g., via inbreeding or selfing). In this context, the term “pedigree” denotes the lineage of a plant, e.g. in terms of the sexual crosses effected such that a gene or a combination of genes, in heterozygous (hemizygous) or homozygous condition, imparts a desired trait to the plant.

As used herein, the term “locus” (plural: “loci”) refers to any site that has been defined genetically. A locus may be a gene, or part of a gene, or a DNA sequence that has some regulatory role, and may be occupied by the same or different sequences.

As used herein, the term “male” refers to a plant that produces pollen grains. The “male plant” generally refers to the sex that produces gametes for fertilizing ova. A plant designated as a “male plant” may contain both male and female sexual organs. Alternatively, the “male plant” may only contain male sexual organs either naturally (e.g., in dioecious species) or due to emasculation (e.g., by removing the ovary).

As used herein, the term “mass selection” refers to a form of selection in which individual plants are selected and the next generation propagated from the aggregate of their seeds.

As used herein, the term “open pollination” refers to a plant population that is freely exposed to some gene flow, as opposed to a closed one in which there is an effective barrier to gene flow.

As used herein, the terms “open-pollinated population” or “open-pollinated variety” refer to plants normally capable of at least some cross-fertilization, selected to a standard, that may show variation but that also have one or more genotypic or phenotypic characteristics by which the population or the variety can be differentiated from others. A hybrid, which has no barriers to cross-pollination, is an open-pollinated population or an open-pollinated variety.

As used herein, the term “ovule” refers to the female gametophyte, whereas the term “pollen” means the male gametophyte.

As used herein, the term “phenotype” refers to the observable characters of an individual cell, cell culture, plant, or group of plants which results from the interaction between that individual's genetic makeup (i.e., genotype) and the environment.

As used herein, the term “recombinant” or “recombinants” refer to a cell, tissue or organism that has undergone transformation with recombinant DNA. The original recombinant is designated as “R0” or “R₀.” Selfing the R₀ produces a first transformed generation designated as “R1” or “R₁.”

The term “plants” or “plant” or grammatical equivalents thereof as used herein is to be construed broadly to include, as well as whole organisms (i.e., plants, also sometimes called whole plants) at any stage of their development, plant cells, plant protoplasts, tissue culture, plant calli, plant embryos or parts of a plant such as roots, root tips, stalk, leaves, flowers, anthers, ears, cobs, husks, silks, and kernels.

As used herein, the term “seed” refers to mature corn kernels produced for the purpose of propagating the species.

As used herein, the term “self pollinated” or “self-pollination” means the pollen of one flower on one plant is applied (artificially or naturally) to the ovule (stigma) of the same or a different flower on the same plant.

As used herein, “MST PCT” refers to the actual moisture of grain at harvest.

As used herein, “PERCENT DROPPED EARS” refers to the percentage of ears of corn that have detached from the plant and fallen to the ground.

As used herein, “PLTPOP” refers to the percentage of plants which have emerged after planting in comparison to the mean percentage of all hybrids in a common test.

As used herein, “staygreen” refers to a measure of plant health that is determined by the percentage of green tissue compared to desiccated brown tissue on the plant at physiological maturity.

As used herein, “drydown” or “dry down” refer to loss of grain moisture over time.

As used herein, “STKLOD PCT” refers to the percentage of plants in which the stalk is broken below the ear node.

As used herein, “TST/WT LB/BU” refers to a measure of the grain weight in pounds for a given bushel volume.

As used herein, the term “synthetic” refers to a set of progenies derived by intercrossing a specific set of clones or seed-propagated lines. A synthetic may contain mixtures of seed resulting from cross-, self-, and/or sib-fertilization.

As used herein, the term “transformation” refers to the transfer of nucleic acid (i.e., a nucleotide polymer) into a cell. As used herein, the term “genetic transformation” refers to the transfer and incorporation of DNA, especially recombinant DNA, into a cell.

As used herein, the term “transformant” refers to a cell, tissue or organism that has undergone transformation. The original transformant is designated as “T0” or “T₀.” Selfing the T0 produces a first transformed generation designated as “T1” or “T₁.”

As used herein, the term “transgenic” refers to cells, cell cultures, organisms, plants, and progeny of plants which have received a foreign or modified gene by one of the various methods of transformation, wherein the foreign or modified gene is from the same or different species than the species of the plant, or organism, receiving the foreign or modified gene.

As used herein, the term “variety” refers to a subdivision of a species, consisting of a group of individuals within the species that are distinct in form or function from other similar arrays of individuals.

S2338 is a cross between the female inbred (ws)LH198 by the male inbred (ws)LH324. Inbreds (ws)LH198 and (ws)LH324 were developed using hybrid and backcross breeding so that each inbred has an amylopectin starch content between about 99% to about 100%.

Hybrid S2338 is characterized by high agronomic yield and average drydown. Waxy maize hybrids are generally agronomically equal to other types of maize hybrids such as dent and waxy grown for commercial production. It is adapted to the east central corn belt regions of Indiana and Ohio. The hybrid has the following characteristics based on data collected from field plots located in Lebanon, Ind., New Ross, Ind., Whiteland, Ind. and Anderson, Ind.

TABLE 1

| A. Type: S2338 | | |
|---|---|---|
| B. Maturity: | | |
| Days | Heat Units | |
| 73 | 1565 | From Plant emergence to 50% of plants with pollen |
| 71 | 1595 | From plant emergence to 50% of plants with silk |
| C. Plant Characteristics | | |
| A. Type: 2 (1 = Sweet 2 = dent 3 = Flint 4 = Flour 5 = Pop) | | |
| Pedigree: **Waxy | | |
| B. Maturity: | | |
| Days | Heat Units | |
| 73_{——} | _1565_{———} | From plant emergence to 50% of plants with pollen |
| 71_{——} | 1595_{———} | From Plant emergence to 50% of plants with silk |

| | | | Standard | | Sample |
|---|---|---|---|---|---|
| | | | Deviation | | Size |
| C. Plant Characteristics: | | | | | |
| 259 | CM | Plant Height (tassel tip) | 6.74 | CM | 25 |
| 66 | CM | Ear Height (base of top ear node) | 3.22 | CM | 25 |
| 0 | | Average number of tillers/plant | 0.11 | | 25 |
| 1 | | Average number of ears/stalk | 0.23 | | 25 |
| | | Root Color | Munsell code: | | 2.5GY 7/6 |
| 1 | | Anthocyanin of brace roots (1 = absent; 2 = faint; 3 = moderate; 4 = dark; 5 = very dark) | | | |
| D. Leaf | | | | | |
| 10.12 | CM | Width of ear node leaf | 1.04 | CM | 25 |
| 81 | CM | Length of ear node leaf | 1.64 | CM | 25 |
| | | Leaf Color | Munsell code: | | 5GY 3/4 |
| 30-60 | Degrees | Leaf Arch | | | |
| E. Tassel | | | | | |
| 7.6 | | Number of primary lateral branches | 1.33 | | 25 |
| 38.5 | CM | Tassel length (top leaf collar to tassel tip) | 2.01 | CM | 25 |
| Sterile | | Pollen shed (1 = light to 9 = heavy) | 0 | | 25 |
| | | Anther color Green | Munsell code: | | 2.5GY 8/6 |
| | | Glume color Pink | Munsell code: | | 5R 8/2 |
| 8.3 | CM | Peduncle length (top leaf to basal branches) | 1.1 | CM | 25 |
| >60 | | Tassel Arch | .16 | | 25 |
| F. Ear (unhusked data) | | | | | |
| Green | | Silk color (3 days after emergence) | Munsell code: | | 2.5GY 8/10 |
| | | Husk cover (25 days after 50% silking) | Munsell code: | | 7.5GY7/2 |
| | | Dry husk cover (65 days after 50% shedding) | Munsell code: | | 2.5Y 8/2 |
| Down | | Position of ear at dry husk stage | | | |
| medium | | Husk tightness | | | |
| 2 | | Husk extension (1 = short (exposed); 2 = medium (<8 cm); 3 = long (8-10 cm beyond | | | |
| | | ear tip); 4 = very long (>10 cm)) | | | |
| G. Ear (husked data) | | | | | |
| 22.8 | CM | Ear length | 2.6 | CM | 25 |
| 5.1 | CM | Ear diameter at midpoint | 0.65 | CM | 25 |
| 292.45 | GR | Ear weight | 9.23 | gr | 25 |
| 14 | | Number of kernel rows | 1.63 | | 25 |
| 1 | | Kernel rows (1 = indistinct; 2 = distinct) | | | |
| 1 | | Row alignment (1 = straight; 2 = slightly curved; 3 = spiral) | | | |
| 2 | | Ear taper (1 = slight; 2 = average; 3 = extreme) | | | |
| H. Kernel (dried) | | | | | |
| 14.87 | MM | Kernel length | 2.01 | MM | 25 |
| 7.64 | MM | Kernel width | 0.54 | MM | 25 |
| 3.77 | MM | Kernel thickness | 0.03 | MM | 25 |
| 3.2% | | % Round kernels | 0.12% | | 25_{——} |
| 1 | | Aleurone color pattern (1 = homozygous; 2 = segregating) | | | |
| 2.5Y 8/8 | | Aleurone color | Munsell code: | | 2.5Y 8/8 |
| | | Hard endosperm color | Munsell code: | | 2.5Y 8/2 |
| | | Endosperm type | Waxy | | |
| 37.69 | gr | Weight per 100 kernels | 1.46 | gr | 25 |
| I. Cob | | | | | |
| 2.63 | CM | Cob diameter at mid-point | 0.67 | CM | 25 |

| | | | |
|---|---|---|---|
| Red | Cob color Red | Munsell code: | 10 R 4/6 |

J. Disease resistance (Rate from 1 = most susceptible to 9 = most resistant)

| | |
|---|---|
| 7_{——} | Common rust (Puccinia sorghi) |
| 6_{——} | Grey leaf spot (Cercospora zeae-maydis) |
| 7_{——} | Northern leaf blight (Exserohilum turcicum) |
| 6_{——} | Southern leaf blight (Bipolaris maydis) |
| 6_{——} | Stewart's wilt (Erwinia stewarti) |

K. Insect resistance

| | |
|---|---|
| | European corn borer (Ostrinia nubalis) |
| _5_{—} | First generation |
| 3_{——} | Second generation |

L. Agronomic traits

| | |
|---|---|
| _7_{—} | Staygreen (70 days after anthesis, rating scale 1-9, 9 = best) |
| _0.042%_{—} | Percent dropped ears (70 days after anthesis) |

Variants, mutants and trivial modifications of the hybrid seed or plant S2338 are within the scope of the present invention. A trivial modification may be a modification of the genetic code of the hybrid plant which results in a plant having at least one, or more, or all of the desirable traits of hybrid S2338, as discussed above, and which preferably has all or substantially all of the morphological or physiological characteristics of the hybrid S2338. In some embodiments, in addition to having all of the morphological and physiological characteristics of S2338, the variant is male sterile. In some embodiments, the male sterility is cytoplasmic male sterility. In some embodiments, the cytoplasmic male sterility is produced by SDms.

Variants, mutants and trivial modifications of the seed or plant of the corn line of the present invention can be generated by methods available to one skilled in the art, including but not limited to, mutagenesis (e.g., chemical mutagenesis, radiation mutagenesis, transposon mutagenesis, insertional mutagenesis, signature tagged mutagenesis, site-directed mutagenesis, and natural mutagenesis), knock-outs/knock-ins, antisense and RNA interference. For more information of mutagenesis in plants, such as agents, protocols, see Acquaah et al. (Principles of plant genetics and breeding, Wiley-Blackwell, 2007, ISBN 1405136464, 9781405136464, which is herein incorporated by reference in its entity).

The invention also relates to a mutagenized population of corn line S2338, and methods of using such populations. In some embodiments, the mutagenized population can be used in screening for new corn lines which comprises one or more or all of the morphological and physiological characteristics of corn line S2338. In some embodiments, the new corn lines obtained from the screening process comprise all of the morphological and physiological characteristics of corn line S2338, and one or more additional or different morphological and physiological characteristics that the corn line S2338 does not have.

The mutagenized population of the present invention can be used in Targeting Induced Local Lesions in Genomes (TILLING) screening method, which combines a standard and efficient technique of mutagenesis with a chemical mutagen (e.g., Ethyl methanesulfonate (EMS)) with a sensitive DNA screening-technique that identifies single base mutations (also called point mutations) in a target gene. Detailed description on methods and compositions on TILLING® can be found in Till et al. (Discovery of induced point mutations in maize genes by TILLING, BMC Plant Biology 2004, 4:12), Weil et al., (TILLING in Grass Species, Plant Physiology January 2009 vol. 149 no. 1 158-164), Comai, L. and S. 1-lenikoff (“TILLING: practical single-nucleotide mutation discovery.” Plant J 45(4): 684-94), McCallum et al., (Nature Biotechnology, 18: 455-457, 2000), McCallum et al., (Plant Physiology, 123: 439-442, 2000), Colbert et al., (Plant Physiol. 126(2): 480-484, 2001), U.S. Pat. No. 5,994,075, U.S. Patent Application Publication No. 2004/0053236A1, and International Patent Application Publication Nos. WO 2005/055704 and WO 2005/048692, each of which is hereby incorporated by reference for all purposes.

It may be preferred that a seed or plant, e.g., a variant seed or plant, according to the present invention has a genome with at least 90%, 91%, 92%, 93%, 94%, 95%, 96%, 97%, 98%, 99%, 99.5% or 99.9% genetic identity with the genome of hybrid.

A progeny plant of hybrid S2338 (in any generation) or a plant derived from hybrid S2338 may preferably have at least 25%, 30%, 35%, 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, 99.5%, 99.9% or 100% genetic identity with hybrid maize plant S2338.

The genotype of a plant and the degree of genetic identity to hybrid S2338 can be assessed using plant breeder records kept routinely by one of ordinary skill in the art. The genotype can additional or alternatively be assessed using molecular marker techniques, e.g., by genetic marker profiling.

A genetic marker profile can be obtained by techniques such as Restriction Fragment Length Polymorphism (RFLP), Randomly Amplified Polymorphic DNA (RAPD), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), Simple Sequence Repeats (SSRs) which are also referred to as microsatellites, and Single Nucleotide Polymorphisms (SNPs). For example see Berry, Don et al “Assessing Probability of Ancestry Using Simple Sequence Repeat Profiles: Applications to Maize Hybrids and Inbreds” Genetics 2002, 161: 813-824.

SSRs are frequently used for mapping purposes. This method is based on repeated sequences which may be repeated a variable number of times at any given locus, thus giving rise to polymorphism, with the potential for multiple alleles. Detection of SSR can be achieved by a number of methods, including PCR. The PCR detection is done using two primers flanking the region containing the repeats (such primers are publicly available). Following amplification, markers can be scored by gel electrophoresis of the amplification products. Scoring of the marker genotype is based on the size of the amplified fragment as measured by molecular weight, rounded to the nearest integer. Relative values should remain constant regardless of the specific primer or precise technique used.

Thus, references to percentage genetic identity may be references to percentage molecular marker profile identity. The molecular marker profile may be an SSR profile. The percentages may refer to the genetic contribution in the molecular marker profile from hybrid S2338.

It may be preferred that a seed or plant according to the present invention has one or more additional desirable traits and/or one or more inserted nucleic acid sequences conferring a desirable trait when compared to hybrid S2338. The nucleic acid sequence may be have been inserted into the seed or plant or any progenitor thereof by any of the methods known to one skilled in the art, e.g., by transgenic techniques or by conventional breeding techniques such as backcrossing. Desirable traits include, but are not limited to, insect, pest or disease resistance, resistance to a herbicide, increased drought or cold resistance, male sterility and modification of the properties of the corn grain (e.g., modified fatty acid metabolism, decreased phytate content, modified carbohydrate composition or the like). The source of the nucleic acid may be a plant of the same or different species, or may be any other organism such as an animal (e.g., an insect), prokaryote, fungus, or a virus. The nucleic acid may also be an artificial nucleic acid, i.e., one not appearing in nature.

Specific examples of such genes would be well known to the skilled person, but some which could be used include a Bacillus thuringiensis protein, a plant disease resistance gene, a lectin, a vitamin binding protein such as avidin, a protease inhibitor or amylase inhibitor, a mutant EPSP or aroA gene, an antisense ACP gene or a phytase encoding gene. The nucleic acids may be any genetic material capable of modifying the plant's phenotype, e.g., conferring or improving a desirable trait, when expressed in a plant, including antisense nucleic acids, siRNAs and the like as well as nucleic acid sequences encoding proteins. The nucleic acid may also be or comprise an enhancer of a promoter. Examples of suitable nucleic acids can be found in U.S. Pat. No. 6,777,598, the disclosure of which is incorporated explicitly by reference.

Transgenic methods are well known to those in the art. Both physical and biological methods for plant transformation are well known in the art (see, for example, Miki et al, “Procedures for Introducing Foreign DNA into Plants”, in Methods in Plant Molecular Biology and Biotechnology, Glick, B. R. and Thompson, J. E. Eds (CRC Press, Inc, Boca Raton, 1993) pages 67-88). Expression vectors and in vitro culture methods for plant cell and tissue transformation and regeneration of plants are also available. See for example Gruber et al “Vectors for Plant Transformation”, in Methods in Plant Molecular Biology and Biotechnology, Glick, B. R. and Thompson, J. E. Eds (CRC Press, Inc, Boca Raton, 1993) pages 89-119, and U.S. Pat. Nos. 6,118,055; 5,405,765; 5,472,869; 5,538,877; 5,538,880; 5,550,318; 5,641,664; 5,736,369 and 5,736,369; 4,940,838; 5,464,763; 5,149,645; 5,501,967; 6,265,638; 4,693,976; 5,635,381; 5,731,179; 5,693,512; 6,162,965; 5,693,512; 5,981,840; 6,420,630; 6,919,494; 6,329,571; 6,215,051; 6,369,298; 5,169,770; 5,376,543; 5,416,011; 5,569,834; 5,824,877; 5,959,179; 5,563,055; and 5,968,830. International Patent Application Publication Nos. WO2002/038779 and WO/2009/117555; Lu et al., (Plant Cell Reports, 2008, 27:273-278); Watson et al., Recombinant DNA, Scientific American Books (1992); Hinchee et al., Bio/Tech. 6:915-922 (1988); McCabe et al., Bio/Tech. 6:923-926 (1988); Toriyama et al., Bio/Tech. 6:1072-1074 (1988); Fromm et al., Bio/Tech. 8:833-839 (1990); Mullins et al., Bio/Tech. 8:833-839 (1990); Hiei et al., Plant Molecular Biology 35:205-218 (1997); Ishida et al., Nature Biotechnology 14:745-750 (1996); Zhang et al., Molecular Biotechnology 8:223-231 (1997); Ku et al., Nature Biotechnology 17:76-80 (1999); and, Raineri et al., Bio/Tech. 8:33-38 (1990)), each of which is expressly incorporated herein by reference in their entirety. Non-limiting examples of vectors that can be used for corn transformation corn transformation are described by Sidorov and Duncan, 2008 (Agrobacterium-Mediated Maize Transformation: Immature Embryos Versus Callus, Methods in Molecular Biology, 526:47-58), Frame et al., 2002 (Agrobacterium tumefaciens-Mediated Transformation of Maize Embryos Using a Standard Binary Vector System, Plant Physiology, May 2002, Vol. 129, pp. 13-22), Ahmadabadi et al., 2007 (A leaf-based regeneration and transformation system for maize (Zea mays L.), TransgenicRes. 16, 437-448), U.S. Pat. Nos. 6,420,630, 6,919,494 and 7,682,829, each of the references above is incorporated herein by reference in its entirety.

The present invention also relates in some aspects and embodiments to tissue cultures, to the use of these cultures and to methods comprising producing plants from these cultures.

Duncan, Williams, Zehr, and Widholm, Planta, (1985)165:322-332 reflects that 97% of the plants cultured which produced callus were capable of plant regeneration. Subsequent experiments with both inbreds and hybrids produced 91% regenerable callus which produced plants. In a further study in 1988, Songstad, Duncan & Widholm in Plant Cell Reports (1988), 7:262-265 reports several media additions which enhance regenerability of callus of two inbred lines. Other published reports also indicated that “nontraditional” tissues are capable of producing somatic embryogenesis and plant regeneration. K. P. Rao, et al., Maize Genetics Cooperation Newsletter, 60:64-65 (1986), refers to somatic embryogenesis from glume callus cultures and B. V. Conger, et al., Plant Cell Reports, 6:345-347 (1987) indicates somatic embryogenesis from the tissue cultures of maize leaf segments. Thus, it is clear from the literature that the state of the art is such that these methods of obtaining plants are, and were, “conventional” in the sense that they are routinely used and have a very high rate of success.

Tissue culture of maize is described in European Patent Application, publication 160,390, incorporated herein by reference. Maize tissue culture procedures are also described in Green and Rhodes, “Plant Regeneration in Tissue Culture of Maize,” Maize for Biological Research (Plant Molecular Biology Association, Charlottesville, Va. 1982, at 367-372) and in Duncan, et al., “The Production of Callus Capable of Plant Regeneration from Immature Embryos of Numerous Zea Mays Genotypes,” 165 Planta 322-332 (1985).

During the production of hybrid seed, effort is made to prevent self pollination of the inbred parent lines. This can be done by conferring male sterility on one of the parent lines by techniques which will be apparent to the skilled person, including the techniques discussed above. However, in the field, complete male sterility of the female parent is extremely difficult to achieve and so in packaged hybrid seed, there is potential for the inclusion of a small amount of the selfed female parent even when the female seed is or has been treated so as to be male sterile. Also, because the male parent is grown next to the female parent in the field there is the possibility that the male selfed seed could be unintentionally harvested and packaged with the hybrid seed.

Therefore, a population of seeds according to the invention may comprise a majority of seeds produced by hybridization of the two parents, and also comprises levels of seed produced from the selfed parent strains (equivalent to the inbred male and female parent lines) that would be expected to result from the normal methods of producing the hybrid. For example, the seed population may comprise at least 90%, 91%, 92%, 93%, 94%, 95%, 96%, 97%, 98%, 99%, 99.5%, 99.9% or 100% of seed produced from the hybridization of the two parents. The amount of the female inbred line (i.e., seed produced from the selfed female parent) may be less than 10%, 9%, 8%, 7%, 6%, 5%, 4%, 3%, 2%, 1%, 0.5%, 0.4%, 0.3%, 0.2%, 0.1% or 0.05%. The amount of the male inbred line (i.e., seed produced from the selfed male parent) may be less than 5%, 4%, 3%, 2%, 1%, 0.5%, 0.4%, 0.3%, 0.2%, 0.1% or 0.05%.

The self-pollinated plants can be identified and distinguished from the hybrid seed because the self-pollinated plants will be genetically equivalent to one of the inbred lines used to produce the hybrid. Due to the level of homozygosity, they will show decreased vigor when compared to the hybrid. For instance, inbreds are identified by their less vigorous appearance for vegetative and/or reproductive characteristics, including shorter plant height, small ear size, ear and kernel shape, cob color, or other characteristics.

Identification of these self-pollinated lines can also be accomplished through molecular marker analyses. See, “The Identification of Female Selfs in Hybrid Maize: A Comparison Using

Electrophoresis and Morphology”, Smith, J. S. C. and Wych, R. D., Seed Science and Technology 14, pp. 1-8 (1995), the disclosure of which is expressly incorporated herein by reference. The inbreds can be identified as being homozygous at one or more loci. See also, “Identification of Atypical Plants in Hybrid Maize Seed by Postcontrol and Electrophoresis” Sarca, V. et al., Probleme de Genetica Teoritica si Aplicata Vol. 20 (1) p. 29-42.

### INDUSTRIAL APPLICABILITY

Corn has extensive use as animal feed, in providing food for human consumption, and in providing raw materials for industry.

Corn, including both grain and non-grain portions, is extensively used as a feed for livestock, such as pigs, cattle and poultry. The grain is also used for human consumption. In addition, corn kernels can be wet milled to produce corn starch, corn syrup and dextrose, or can be dry milled to produce corn flour, grits and meal. Corn oil is recovered from corn germ, which is a by-product of both the wet and dry milling industries.

Uses of corn starch are based on functional properties such as viscosity, film formation, adhesive properties and the ability to suspend particles. Corn starch can be used in industry in the production of paper, textiles and adhesives. It is also useful in building materials, foundry binders, laundry starches, explosives, oil-well muds, oil-drilling fluids and other mining applications. Due to their biodegradable and renewable nature, starches are increasingly being used many other products, including packaging, plastics, detergents, pharmaceutical tablets, pesticides and cosmetics. Starch can also be fermented into ethanol and can also be processed into corn syrups and sweeteners such as high fructose corn syrup and dextrose. Starch can be used in an unmodified or modified form (e.g., acid modified corn starch, dextrins, oxidized corn starch, pregelatinized starch and chemically derivatized starch).

Corn starch is made up of two components, amylose and amylopectin. Amylose consists of predominantly linear chains of glucose monomers linked by 1,4-glycosidic bonds. In amylopectin, the chains are branched by the addition of 1,6-glycosidic bonds. Starches and flours having different proportions of amylose and amylopectin are particularly adapted to different industrial purposes.

High amylose starch may be recognized by one or more of the following properties. The granules are of two distinct types, spherical and irregular, and are smaller than normal starch granules. The Birefringence End Point Temperature (“BEPT”) is reported as 97° C. BEPT is the temperature at which the starch molecule loses organized structure. Some of the granules do not lose all birefringence even after prolonged boiling; swelling power is only about one-fourth and solubles about one-half that of regular corn starch at 95° C. (Corn and Corn Improvement, third edition, Ed. Sprague and Dudley).

High-amylose starches are particularly useful in confectionery such as gummed candies (because they thicken rapidly), in fried snacks (because they resist the penetration of cooking oil), and in photographic film (because of their toughness and transparency), as well as in the uses discussed above (e.g., textiles, biodegradable packaging materials, adhesives for manufacturing corrugated cardboard, and the like). It has also been suggested that the anti-staling properties of bread can be improved by the use of flour high in amylose. Other uses include the sizing of glass fibers prior to weaving, the preparing of a clear, hot water dispersible, edible film for packaging food, dyes and other soluble materials, and coating paper to reduce water and fat absorption. Nutritional aspects are primarily what we are developing with high amylose starches, particularly high fiber, high resistance to digestion, low calorie, and control of glycemic response.

Amylopectin is particularly useful in paper-making and adhesives (because its branched chains give it greater binding power), and in ready prepared foods, such as in frozen and canned food (because it enhances stability and shelf-life), and fruit pie fillings (where it acts as a thickener). It is useful for the formation of translucent films which are readily redissolved, as well as the uses discussed above.

Other uses of corn include the use of stalks and husks for paper and wall board and the use of cobs for fuel, to make charcoal and for the production of fufural.

### Breeding Methods

Classic breeding methods can be included in the present invention to introduce one or more recombinant expression cassettes of the present invention into other plant varieties, or other close-related species that are compatible to be crossed with the transgenic plant of the present invention.

Open-Pollinated Populations.

The improvement of open-pollinated populations of such crops as rye, many maizes and sugar beets, herbage grasses, legumes such as alfalfa and clover, and tropical tree crops such as cacao, coconuts, oil palm and some rubber, depends essentially upon changing gene-frequencies towards fixation of favorable alleles while maintaining a high (but far from maximal) degree of heterozygosity. Uniformity in such populations is impossible and trueness-to-type in an open-pollinated variety is a statistical feature of the population as a whole, not a characteristic of individual plants. Thus, the heterogeneity of open-pollinated populations contrasts with the homogeneity (or virtually so) of inbred lines, clones and hybrids.

Population improvement methods fall naturally into two groups, those based on purely phenotypic selection, normally called mass selection, and those based on selection with progeny testing. Interpopulation improvement utilizes the concept of open breeding populations; allowing genes to flow from one population to another. Plants in one population (cultivar, strain, ecotype, or any germplasm source) are crossed either naturally (e.g., by wind) or by hand or by bees (commonly Apis mellifera L. or Megachile rotundata F.) with plants from other populations. Selection is applied to improve one (or sometimes both) population(s) by isolating plants with desirable traits from both sources.

There are basically two primary methods of open-pollinated population improvement. First, there is the situation in which a population is changed en masse by a chosen selection procedure.

The outcome is an improved population that is indefinitely propagable by random-mating within itself in isolation. Second, the synthetic variety attains the same end result as population improvement but is not itself propagable as such; it has to be reconstructed from parental lines or clones. These plant breeding procedures for improving open-pollinated populations are well known to those skilled in the art and comprehensive reviews of breeding procedures routinely used for improving cross-pollinated plants are provided in numerous texts and articles, including: Allard, Principles of Plant Breeding, John Wiley & Sons, Inc. (1960); Simmonds, Principles of Crop Improvement, Longman Group Limited (1979); Hallauer and Miranda, Quantitative Genetics in Maize Breeding, Iowa State University Press (1981); and, Jensen, Plant Breeding Methodology, John Wiley & Sons, Inc. (1988).

Mass Selection.

In mass selection, desirable individual plants are chosen, harvested, and the seed composited without progeny testing to produce the following generation. Since selection is based on the maternal parent only, and there is no control over pollination, mass selection amounts to a form of random mating with selection. As stated herein, the purpose of mass selection is to increase the proportion of superior genotypes in the population.

Synthetics.

A synthetic variety is produced by crossing inter se a number of genotypes selected for good combining ability in all possible hybrid combinations, with subsequent maintenance of the variety by open pollination. Whether parents are (more or less inbred) seed-propagated lines, as in some sugar beet and beans (Vicia) or clones, as in herbage grasses, clovers and alfalfa, makes no difference in principle. Parents are selected on general combining ability, sometimes by test crosses or toperosses, more generally by polycrosses. Parental seed lines may be deliberately inbred (e.g. by selfing or sib crossing). However, even if the parents are not deliberately inbred, selection within lines during line maintenance will ensure that some inbreeding occurs. Clonal parents will, of course, remain unchanged and highly heterozygous.

Whether a synthetic can go straight from the parental seed production plot to the farmer or must first undergo one or two cycles of multiplication depends on seed production and the scale of demand for seed. In practice, grasses and clovers are generally multiplied once or twice and are thus considerably removed from the original synthetic.

While mass selection is sometimes used, progeny testing is generally preferred for polycrosses, because of their operational simplicity and obvious relevance to the objective, namely exploitation of general combining ability in a synthetic.

The number of parental lines or clones that enter a synthetic vary widely. In practice, numbers of parental lines range from 10 to several hundred, with 100-200 being the average. Broad based synthetics formed from 100 or more clones would be expected to be more stable during seed multiplication than narrow based synthetics.

Pedigreed Varieties.

A pedigreed variety is a superior genotype developed from selection of individual plants out of a segregating population followed by propagation and seed increase of self pollinated offspring and careful testing of the genotype over several generations. This is an open pollinated method that works well with naturally self pollinating species. This method can be used in combination with mass selection in variety development. Variations in pedigree and mass selection in combination are the most common methods for generating varieties in self pollinated crops.

Hybrids.

A hybrid is an individual plant resulting from a cross between parents of differing genotypes. Commercial hybrids are now used extensively in many crops, including corn (maize), sorghum, sugarbeet, sunflower and broccoli. Hybrids can be formed in a number of different ways, including by crossing two parents directly (single cross hybrids), by crossing a single cross hybrid with another parent (three-way or triple cross hybrids), or by crossing two different hybrids (four-way or double cross hybrids).

Strictly speaking, most individuals in an out breeding (i.e., open-pollinated) population are hybrids, but the term is usually reserved for cases in which the parents are individuals whose genomes are sufficiently distinct for them to be recognized as different species or subspecies. Hybrids may be fertile or sterile depending on qualitative and/or quantitative differences in the genomes of the two parents. Heterosis, or hybrid vigor, is usually associated with increased heterozygosity that results in increased vigor of growth, survival, and fertility of hybrids as compared with the parental lines that were used to form the hybrid. Maximum heterosis is usually achieved by crossing two genetically different, highly inbred lines.

The production of hybrids is a well-developed industry, involving the isolated production of both the parental lines and the hybrids which result from crossing those lines. For a detailed discussion of the hybrid production process, see, e.g., Wright, Commercial Hybrid Seed Production 8:161-176, In Hybridization of Crop Plants.

### EXAMPLES

It should be understood that the examples and embodiments described herein are for illustrative purposes only and that various modifications or changes in light thereof will be suggested to persons skilled in the art and are to be included within the spirit and purview of this application.

### Example 1

### Hybrid Comparisons for Agronomic Traits

In yield testing, the male-sterile S2338 plants created by crossing wsLH198SDms×wsLH324 were evaluated and two waxy pollinators were interspersed throughout the small plot yield evaluation to provide a bulk pollen cloud. The plants created by this process are designated as S2338HCT. Comparisons of the agronomic characteristics of S2338HCT were made to S2994HCT which is the commercial waxy hybrid produced in a similar way as S2338HCT and has a similar maturity and adaptation, over a three-year period at three locations. The comparison is provided in Table 2 below.

TABLE 2
Yield Comparison

| | Number | | | | |
|---|---|---|---|---|---|
| | of plants | | | | |
| | (3 yrs, 3 | Average | Average | Average | Average |
| Hybrid | locations) | BuA | Mst % | Twt | Pop |
| S2944HCT | 130 | 175 | 23 | 56 | 54 |
| [(ws)FR1064SDms × | | | | | |
| (ws)LH185] × | | | | | |
| fertile waxy pollinators | | | | | |
| S2338HCT | 123 | 184 | 22 | 58 | 58 |
| [(ws)LH198SDms × | | | | | |
| (ws)LH324] × | | | | | |
| fertile waxy pollinators | | | | | |

BUA—Bushels/Acre
MST—Harvest Grain Moisture
TWT—Grain Test Weight
POP—Stand Population, number of plants in 2 rows(17.5 ft)

Further comparisons of the agronomic characteristics of S2338HCT were made to S2944HCT over a four year period at 5 locations in Indiana. The comparison is provided in Table 3.

TABLE 3
Hybrid Yield Summary Data for S2338HCT and S2944HCT.
Comparison data represent averages of yield trials conducted over
a 4 year period at 5 locations in Indiana. (Bpa = bushels per acre)
S2338HCT WaxiPro ® Yield Data

| Year | Variety | Location | BPA | Moist |
|---|---|---|---|---|
| Year 1 | S2338HCT | Lebanon | 194.53 | 16.20 |
| | S2338HCT | New Ross | 196.74 | 16.10 |
| | S2338HCT | Anderson | 201.43 | 15.80 |
| | S2338HCT | Franklin | 185.45 | 15.90 |
| | S2338HCT | Monrovia | 198.63 | 16.10 |
| | S2944HCT | Lebanon | 190.20 | 16.80 |
| | S2944HCT | New Ross | 188.64 | 16.70 |
| | S2944HCT | Anderson | 190.32 | 15.90 |
| | S2944HCT | Franklin | 179.41 | 15.80 |
| | S2944HCT | Monrovia | 182.52 | 16.50 |
| Year 2 | S2338HCT | Lebanon | 203.14 | 16.40 |
| | S2338HCT | New Ross | 202.31 | 16.80 |
| | S2338HCT | Anderson | 203.41 | 17.40 |
| | S2338HCT | Franklin | 198.70 | 16.10 |
| | S2338HCT | Monrovia | 199.35 | 16.30 |
| | S2944HCT | Lebanon | 194.20 | 15.90 |
| | S2944HCT | New Ross | 193.64 | 15.80 |
| | S2944HCT | Anderson | 195.45 | 16.90 |
| | S2944HCT | Franklin | 189.40 | 16.80 |
| | S2944HCT | Monrovia | 188.60 | 16.40 |
| Year 3 | S2338HCT | Lebanon | 199.65 | 15.80 |
| | S2338HCT | New Ross | 198.78 | 15.60 |
| | S2338HCT | Anderson | 200.14 | 16.10 |
| | S2338HCT | Franklin | 199.56 | 15.90 |
| | S2338HCT | Monrovia | 199.65 | 16.40 |
| | S2944HCT | Lebanon | 190.30 | 15.80 |
| | S2944HCT | New Ross | 190.30 | 15.70 |
| | S2944HCT | Anderson | 191.45 | 16.40 |
| | S2944HCT | Franklin | 189.45 | 16.10 |
| | S2944HCT | Monrovia | 190.33 | 15.90 |
| Year 4 | S2338HCT | Lebanon | 154.42 | 14.30 |
| | S2338HCT | New Ross | 150.38 | 14.70 |
| | S2338HCT | Anderson | 161.23 | 13.20 |
| | S2338HCT | Franklin | 150.23 | 14.70 |
| | S2338HCT | Monrovia | 154.47 | 14.90 |
| | S2944HCT | Lebanon | 145.60 | 15.30 |
| | S2944HCT | New Ross | 142.31 | 14.80 |
| | S2944HCT | Anderson | 152.67 | 14.70 |
| | S2944HCT | Franklin | 141.70 | 15.10 |
| | S2944HCT | Monrovia | 142.90 | 13.40 |
| Year 1- | S2338HCT | bpa Yield | 187.61 | Average |
| Year 4 | | | | |
| Year 1- | S2944HCT | bpa Yield | 178.47 | Average |
| Year 4 | | | | |
| | | | 9.14 | Difference |
| Year 1 | S2338HCT | bPA Yield | 195.36 | Average |
| Year 1 | S2944HCT | bpa Yield | 186.22 | Average |
| | | | 9.14 | Difference |
| Year 2 | S2338HCT | bpa Yield | 201.38 | Average |
| Year 2 | S2944HCT | bpa Yield | 192.26 | Average |
| | | | 9.12 | Difference |
| Year 3 | S2338HCT | bpa Yield | 199.56 | Average |
| Year 3 | S2944HCT | bpa Yield | 190.37 | Average |
| | | | 9.19 | Difference |
| Year 4 | S2338HCT | bpa Yield | 154.15 | Average |
| Year 4 | S2944HCT | bpa Yield | 145.04 | Average |
| | | | 9.11 | Difference |

As shown in Tables above, S2338HCT has significantly higher agronomic yield than S2944HCT. Grain moisture at harvest was comparable indicating that the hybrids have comparable maturities.

### DEPOSIT INFORMATION

A sample of the hybrid corn seed of S2338 has been or will be deposited with the American Type Culture Collection, 10801 University Boulevard, Manassas, Va. 20110 under the Budapest Treaty.

To satisfy the enablement requirements of 35 U.S.C. 112, and to certify that the deposit of the present invention meets the criteria set forth in 37 CFR 1.801-1.809, Applicants hereby make the following statements regarding the deposited corn hybrid line S2338 (deposited as ATCC Accession No. ______):

1. During the pendency of this application, access to the invention will be afforded to the Commissioner upon request;
2. Upon granting of the patent the deposit will be available to the public under conditions specified in 37 CFR 1.808;
3. The deposit will be maintained in a public repository for a period of 30 years or 5 years after the last request or for the effective life of the patent, whichever is longer;
4. The viability of the biological material at the time of deposit will be tested (see 37 CFR 1.807); and
5. The deposit will be replaced if it should ever become unavailable.

Access to this deposit will be available during the pendency of this application to persons determined by the Commissioner of Patents and Trademarks to be entitled thereto under 37 C.F.R. §1.14 and 35 U.S.C. §122. Upon granting of any claims in this application, all restrictions on the availability to the public of the variety will be irrevocably removed by affording access to a deposit of at least 2,500 seeds of the same variety with the ATCC.

Unless defined otherwise, all technical and scientific terms herein have the same meaning as commonly understood by one of ordinary skill in the art to which this invention belongs. Although any methods and materials, similar or equivalent to those described herein, can be used in the practice or testing of the present invention, the non-limiting exemplary methods and materials are described herein.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference. Nothing herein is to be construed as an admission that the present invention is not entitled to antedate such publication by virtue of prior invention.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

What is claimed:

1. Seed of hybrid maize designated S2338, a representative sample of which has been deposited under ATCC Accession Number ______.

2. A maize plant, or part thereof, obtainable by growing the seed of claim 1.

3. The maize plant, or part thereof, of claim 2, wherein the plant, or part thereof, have been transformed so that its genetic material contains one or more transgenes operably linked to one or more regulatory elements.

4. A maize plant having all of the morphological and physiological characteristics of the plant of claim 2.

5. A cytoplasmic male-sterile maize plant having the plant genome of the maize plant of claim 2.

6. A maize plant having all of the morphological and physiological characteristics of the plant of claim 5.

7. A plant part having all of the morphological and physiological characteristics of the plant part of claim 2.

8. A tissue culture of regenerable cells produced from the plant, or part thereof, of claim 2.

9. A maize plant regenerated from a tissue culture of the plant, or part thereof, of claim 2.

10. An ovule of the plant of claim 2.

11. Pollen of a plant of claim 2.

12. A method for producing maize seed comprising crossing the maize plant of claim 2 with itself or another maize plant, and harvesting the resultant seed.

13. The method of claim 12, further comprising growing the resultant seed to produce one or more progeny maize plants, breeding from one or more of said progeny maize plants to produce progeny seed, and harvesting said progeny seed.

14. The method of claim 13, further comprising growing said progeny seed, breeding from the resultant maize plants to produce seed, and harvesting said seed, over 1, 2, 3, 4, 5, 6 or more generations.

15. A seed which when grown produces the plant of claim 4.

16. A method of producing a processed corn product comprising providing at least one plant part of claim 2 and processing said part(s) to produce a processed corn product.

17. The method of claim 16, wherein the part is one or more corn kernels.

18. The method of claim 16, wherein said processed corn product is corn starch.

19. The method of claim 16, wherein said processed corn product is corn flour.

20. A processed corn product produced by the method of claim 16.

21. A method comprising providing the processed corn product of claim 20, and further comprising using said processed corn product in the production of a manufactured product.

22. The method of claim 21, wherein the manufactured product is selected from the list consisting of a confectionery, a fried food product and a baked food product.

21. A method for producing a maize plant derived from hybrid S2338 comprising:
a) crossing a hybrid maize S2338 plant with a second maize plant and harvesting the resultant maize seed, wherein representative seed of S2338 has been deposited under ATCC Accession Number ______; and,
b) growing said resultant maize seed to produce a maize plant derived from S2338.

22. A method for developing a maize plant in a plant breeding program using plant breeding techniques comprising employing a maize plant, or its part, as a source of plant breeding material comprising using the maize plant, or its part, of claim 2 as a source of breeding material.

23. A method for developing a second maize plant in a plant breeding comprising applying plant breeding techniques to a first maize plant, or parts thereof, wherein said first maize plant is the maize plant of claim 4, and wherein application of said techniques results in development of said second maize plant.

24. The method for developing a second maize plant in a maize plant breeding program of claim 23 wherein said plant breeding techniques are selected from the group consisting of pedigree breeding, recurrent selection, backcrossing, restriction fragment length polymorphism enhanced selection, genetic marker enhanced selection and transformation.

25. A method for developing a second maize plant in a plant breeding comprising applying plant breeding techniques to a first maize plant, or parts thereof, wherein said first maize plant is the maize plant of claim 5, and wherein application of said techniques results in development of said second maize plant.

26. A method of producing hybrid maize seed comprising crossing a male-sterile version of S2338 with at least one waxy pollinator hybrid and harvesting the resultant seed from the male-sterile version of S2338.

27. The method of claim 26, wherein the male-sterile version of S2338 is crossed with two waxy pollinator hybrids.

28. The method of claim 26 further comprising growing a hybrid maize plant from the hybrid maize seed harvested from the male-sterile version of S2338.
